(19) United States
(12) Patent Application Publication
(10) Pub. No.: US 2016/0333283 A1
(43) Pub. Date: Nov. 17, 2016

(54) FUEL REFORMING SYSTEM

(71) Applicant: HONDA MOTOR CO., LTD., Tokyo (JP)

(72) Inventors: Kohtaro HASHIMOTO, Wako (JP); Tomohide KUDO, Wako (JP)

(73) Assignee: HONDA MOTOR CO., LTD., Tokyo (JP)

(21) Appl. No.: 15/089,594

(22) Filed: Apr. 4, 2016

(30) Foreign Application Priority Data
May 12, 2015 (JP) 2015-097543

(51) Int. Cl. C10L 1/183; C07C 37/56; C07C 37/82; C10L 10/10

(52) U.S. Cl. CPC C10L 1/1832; C10L 10/10; C07C 37/56; C07C 37/82; C10L 2290/547; C10L 2290/54

(57) ABSTRACT

A fuel reforming system to reform a fuel to be supplied to an internal combustion engine includes a reformer, a first filter, and a first flow path. The reformer includes a reforming catalyst to reform a fuel including a hydrocarbon to produce an alcohol and benzoic acid as a by-product using air. The first filter includes a sodium catalyst to trap the benzoic acid produced by the reforming catalyst. The alcohol produced by the reforming catalyst passes through the first filter. Air passes through the first filter to reform the benzoic acid to phenol. The phenol is mixed with the alcohol produced by the reforming catalyst via the first flow path. Alternatively, the phenol is directly supplied to the internal combustion engine as a fuel via the first flow path.

### CROSS-REFERENCE TO RELATED APPLICATIONS

The present application claims priority under 35 U.S.C. §119 to Japanese Patent Application No. 2015-097543, filed May 12, 2015, entitled “Fuel Reforming System.” The contents of this application are incorporated herein by reference in their entirety.

### BACKGROUND

1. Field of the Invention

The present disclosure relates to a fuel reforming system.

2. Discussion of the Background

It has been known that in a gasoline engine in which a mixture of fuel and air prepared in advance is combusted by spark ignition using a spark plug, an unburnt mixture gas (end gas) remote from the spark plug is self-ignited to cause knocking. Since the knocking is liable to occur when the compression ratio of the engine is increased, in a recent high compression ratio engine, reduction of knocking has been strongly required.

As a method to reduce the knocking, for example, a method in which an ignition timing is retarded may be mentioned. However, when the ignition timing is retarded, a thermal efficiency of the engine is decreased. Hence, for a high compression ratio engine, technical development of realizing a high thermal efficiency besides the reduction of knocking has also been desired.

Incidentally, since the knocking can be reduced by increasing the fuel's octane number, as a fuel having a high octane number, an alcohol-containing fuel in which an alcohol, such as ethanol, is mixed in gasoline in advance has been widely used in some limited areas. In addition, in accordance with the spread of an alcohol-containing fuel as described above, a technique in which an alcohol-containing fuel supplied from the outside is separated into a fuel having a high gasoline concentration and a fuel having a high alcohol concentration on a vehicle has been developed. The reason for this is as follows. Since gasoline is different from an alcohol in terms of fuel properties, such as the octane number and the calorific value, instead of using an alcohol-containing fuel supplied from the outside without modification, an alcohol and gasoline are preferably separated from each other on a vehicle so as to be used in accordance with respective applications. However, the separation in accordance with the requirement of the engine is not easily performed.

In addition, a technique has been proposed in which a plurality of fuels, that is, a low octane number fuel and a high octane number fuel, are stored in advance in raw fuel tanks and are supplied through a plurality of fuel injection units into a cylinder at a mixing ratio in accordance with the operation condition (see, for example, Japanese Unexamined Patent Application Publication No. 2005-54610). According to the technique disclosed in Japanese Unexamined Patent Application Publication No. 2005-54610, the flow rate of each fuel supplied to the corresponding fuel injection unit is detected by a fuel flow rate detector, and based on the flow rate of each fuel thus detected, an actual mixing ratio of the fuels to be supplied into the cylinder is calculated using an actual mixing ratio calculation unit. According to the technique described above, the actual mixing ratio of the fuels to be supplied into the cylinder can be accurately calculated based on the flow rates of the fuels supplied to the respective fuel injection units, each of which is detected by the fuel flow rate detector.

In addition, there has also been proposed a technique in which after a second fuel (such as phenol) having a high octane number is produced from a first fuel (such as gasoline) using a reformer, the first fuel and the second fuel are supplied to an internal combustion engine for operation thereof while the mixing ratio therebetween is adjusted (see, for example, Japanese Unexamined Patent Application Publication No. 2012-184749). According to this proposal, the reformer includes a first reforming portion and a second reforming portion sequentially disposed at a downstream side thereof. By the first reforming portion, a monocyclic aromatic carboxylic acid is produced from a monocyclic aromatic hydrocarbon, and by the second reforming portion, a monocyclic phenol is further produced from the monocyclic aromatic carboxylic acid. According to the technique disclosed in Japanese Unexamined Patent Application Publication No. 2012-184749, since the first fuel and the second fuel can be used at a mixing ratio in accordance with the load required by the internal combustion engine, for example, in view of effective use of resources, the degree of freedom of selecting a high octane number fuel for the internal combustion engine can be increased.

### SUMMARY

According to one aspect of the present invention, a fuel reforming system which reforms a fuel to be supplied into an internal combustion engine, includes: a reformer containing a reforming catalyst which reforms a fuel primarily formed of a hydrocarbon using air to produce an alcohol as a main product and benzoic acid as a by-product; at least one filter which includes a sodium catalyst, which is provided at a downstream side of the reformer, and which allows the alcohol as the main product produced by the reforming catalyst to pass therethrough and traps the benzoic acid precipitated as the by-product; and at least one flow path which merges phenol with the main product produced by the reformer or at least one flow path which directly supplies phenol into the internal combustion engine as a fuel, the phenol being produced by reforming of the trapped benzoic acid with air which is allowed to pass through the filter.

According to another aspect of the present invention, a fuel reforming system to reform a fuel to be supplied to an internal combustion engine includes a reformer, a first filter, and a first flow path. The reformer includes a reforming catalyst to reform a fuel including a hydrocarbon to produce an alcohol and benzoic acid as a by-product using air. The first filter includes a sodium catalyst to trap the benzoic acid produced by the reforming catalyst. The alcohol produced by the reforming catalyst passes through the first filter. Air passes through the first filter to reform the benzoic acid to phenol. The phenol is mixed with the alcohol produced by the reforming catalyst via the first flow path. Or the phenol is directly supplied to the internal combustion engine as a fuel via the first flow path.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings.

FIG. 1 is a schematic view showing the structure of a fuel reforming system according to one embodiment of the present disclosure.

FIG. 2 is a schematic view showing one example of the structure of a filter mechanism applied to the fuel reforming system shown in FIG. 1.

FIG. 3 is a flowchart showing a filter regeneration operation performed when a filter is regenerated in the filter mechanism shown in FIG. 2.

FIG. 4 is a schematic view showing another example of the filter mechanism applied to the fuel reforming system shown in FIG. 1.

### DESCRIPTION OF THE EMBODIMENTS

The embodiments will now be described with reference to the accompanying drawings, wherein like reference numerals designate corresponding or identical elements throughout the various drawings.

One embodiment of the present disclosure will be described in detail with reference to the drawings.

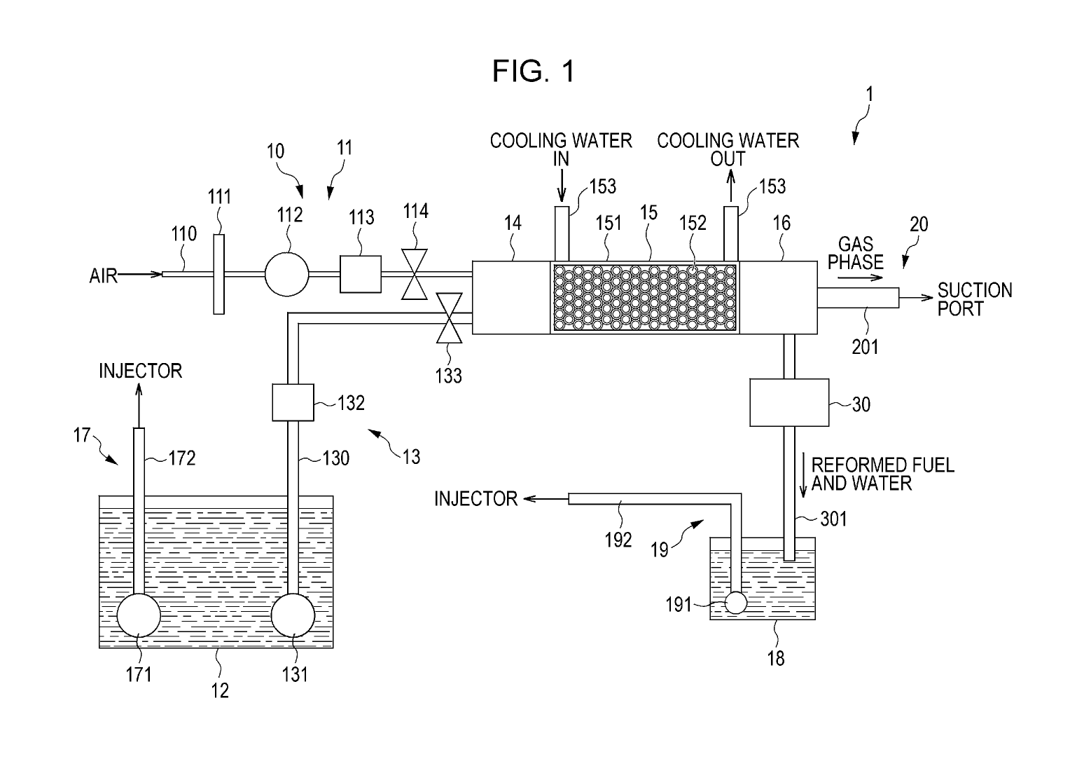

FIG. 1 is a schematic view showing the structure of a fuel reforming system 1 according to one embodiment of the present disclosure. The fuel reforming system 1 according to this embodiment is mounted on a vehicle not shown in the drawing, and in response to the requirement of an internal combustion engine (hereinafter referred to as “engine”) not shown in the drawing, on the vehicle, the fuel reforming system 1 reforms a hydrocarbon contained in a fuel into an alcohol and supplies the alcohol to the engine.

In the fuel reforming system 1 according to this embodiment, gasoline is used as the fuel, and as an oxidizing agent, air is used. That is, in the fuel reforming system 1 according to this embodiment, since gasoline is reformed by the use of an oxidation reaction with oxygen in the air, for example, compared to reforming using a decomposition reaction or the like, reforming may be performed at a low temperature and under mild conditions, and hence, the system structure can be simplified. Accordingly, the fuel reforming system 1 according to this embodiment is suitably applied to on-demand operation on a vehicle.

As shown in FIG. 1, the fuel reforming system 1 according to this embodiment includes an air introduction portion 11, a fuel tank 12, a fuel introduction portion 13, a mixer 14, a reformer 15, a condenser 16, a fuel supply portion 17, a reformed fuel tank 18, a reformed fuel supply portion 19, a gas phase supply portion 20, and a filter mechanism 30.

The air introduction portion 11 is provided at an upstream side of the mixer 14 which will be described later and introduces air as an oxidizing agent into the mixer 14.

The air introduction portion 11 includes an air filter 111, an air pump 112, an air flow meter 113, and an air valve 114 in this order from an upstream side of an air introduction pipe 110.

The air introduction portion 11 introduces air thereinto from the outside through the air filter 111 by driving the air pump 112. In addition, the air introduction portion 11 supplies the above introduced air into the mixer 14 by opening the air valve 114.

Based on the air flow rate detected by the air flow meter 113, by an electronic control unit (hereinafter referred to as “ECU”) not shown in the drawing, the degree of opening of the air valve 114 is adjusted, and in accordance with this degree of opening thereof, the amount of air to be introduced into the mixer 14 is controlled.

The fuel supply portion 17 includes a fuel pump 171, a fuel supply pipe 172, and an injector not shown in the drawing. By driving the fuel pump 171, the fuel supply portion 17 supplies a gasoline stored in the fuel tank 12 to a cylinder of the engine not shown in the drawing through the fuel supply pipe 172 and the injector.

The supply amount of the gasoline to the engine is controlled by adjusting an injection amount of the injector using the ECU.

The fuel introduction portion 13 is provided at an upstream side of the mixer 14 which will be described later and introduces the gasoline functioning as a fuel into the mixer 14.

The fuel introduction portion 13 includes a reforming pump 131, a fuel flow meter 132, and fuel valve 133 in this order from an upstream side of a fuel introduction pipe 130.

The fuel introduction portion 13 introduces the gasoline stored in the fuel tank 12 into the mixer 14 by driving the reforming pump 131 and simultaneously by opening the fuel valve 133.

Based on the fuel flow rate detected by the fuel flow meter 132, the degree of opening of the fuel valve 133 is adjusted by the ECU, and by this adjustment of the degree of opening, the amount of the gasoline to be introduced into the mixer 14 is controlled.

In this fuel reforming system 1, a supply system 10 supplying air and a fuel into the mixer 14 is formed from the air introduction portion 11 and the fuel introduction portion 13.

In the supply system 10, the air introduction portion 11 and the fuel introduction portion 13 are synchronously operated under the ECU control, and the air and the fuel to be supplied to the mixer 14 are adjusted so that with respect to the total amount of the air and the fuel, the rate of the fuel is 22 percent by mass or more.

Since the adjustment described above is performed, the rate of the fuel with respect to the total amount of the air and the fuel to be supplied to the mixer 14 is set to 22 percent by mass or more. This rate corresponds to a fuel-rich region than the explosion limit. Hence, the probability of occurrence of an excessively rapid reaction is minimized, and hence, a conversion process converting the gasoline into an alcohol is made stable.

The mixer 14 is provided at an upstream side of the reformer 15 which will be described later, and after the gasoline functioning as a fuel and the air are mixed together as described above, the mixer 14 supplies the mixture thus obtained into the reformer 15.

This mixer 14 is configured so that the air and the gasoline functioning as a fuel, each of which is to be supplied, are uniformly mixed together.

In addition, the upper limit of the rate of the fuel (gasoline) to the total amount of the air and the fuel described above is approximately 80 percent by mass. When the rate described above is more than this upper limit, oxidation reforming is not likely to occur.

In this embodiment, in the mixer 14, particulate substances or porous substances are provided. By those particulate substances or porous substances, the air and the fuel supplied from the supply system 10 are uniformly mixed together by the generation of dispersion, whirligig, and rotation (revolution) of the flows thereof.

In this embodiment, in particular, the space formed between the particulate substances or the porous substances is 1 mm or less.

As the particulate substance, for example, quartz sand, silicon dioxide, or zeolite may be used. In addition, as the porous substance, for example, porous stainless steel which is sintered stainless steel or other various porous metals may be used.

In addition, the mixer 14 also includes a heater not shown in the drawing, and since mixing is performed while the gasoline and the air are heated to a predetermined temperature, a mixture of the gasoline and the air is produced.

The reformer 15 produces an alcohol by reforming a hydrocarbon which is a main component of the gasoline in the mixture supplied from the mixer 14 using the air in the mixture. In particular, as the reformer 15, either a flow reactor or a completely-mixed reactor may be used.

In this embodiment, the flow reactor is a reactor in which the mixture of the gasoline and the air supplied from the mixer 14 is reformed while flowing through the reactor like a piston without being mixed with mixtures which are supplied before and after the mixture described above in the reactor. In this flow reactor, the composition of a fluid flowing out of the reactor is different from that of a fluid in the reactor, and the variation in residence time of the mixture in the reactor is characteristically small.

On the other hand, the completely-mixed reactor is a reactor in which the mixture of the gasoline and the air supplied from the mixer 14 is uniformly mixed with reaction products in the reactor and is reformed. By this completely-mixed reactor, the composition of a fluid flowing out of the reactor is the same as that of a fluid in the reactor, and the variation in residence time of the mixture in the reactor is characteristically large.

In the fuel reforming system 1 shown in FIG. 1, the reformer 15 is provided with a temperature sensor (not shown) and a cooling portion 153 cooling the inside of the reformer 15. The cooling portion 153 is controlled by the ECU based on the temperature detected by the temperature sensor and cools the reformer 15 by supplying cooling water of the engine to the reformer 15.

The temperature of the engine cooling water is preferably 70° C. to 100° C. When the temperature of the engine cooling water is less than 70° C., the reforming reaction rate is low, and when the temperature is more than 100° C., the engine cooling water is difficult to be used. In addition, when the temperature inside the reformer 15 is increased high as the reforming reaction proceeds, the cooling portion 153 cools the reformer 15 using the engine cooling water; however, when the temperature inside the reformer 15 is low since the reforming reaction is at the initial stage, reversely, the cooling portion 153 functions to warm the reformer 15 using the engine cooling water.

In addition, the reformer 15 includes a reforming catalyst 152 which reforms a hydrocarbon primarily contained in gasoline using air functioning as an oxidizing agent to produce an alcohol. In particular, the reformer 15 includes a cylindrical casing 151 and a solid reforming catalyst 152 filled therein.

The solid reforming catalyst 152 is formed of small ball-shaped porous carriers, a primary catalyst, and an auxiliary catalyst, those two catalysts being carried on the surfaces of the porous carriers. The primary catalyst and the auxiliary catalyst placed in a uniformly mixed state are carried on the surfaces of the small ball-shaped porous carriers. In the reforming catalyst 152 of this embodiment, since the porous carrier has a ball shape as described above, the surface area of the primary catalyst and that of the auxiliary catalyst carried on the surfaces of the porous carriers are increased, and as a result, the contact areas of the catalysts with the gasoline functioning as a fuel and the air functioning as an oxidizing agent are increased.

As the small ball-shaped porous carriers, for example, silica beads, alumina beads, and silica-alumina beads may be used. Among those mentioned above, silica beads are preferably used. The particle diameter of the porous carriers is preferably 3 to 500 μm.

The primary catalyst functions so as to produce an alkyl radical by abstracting a hydrogen atom from a hydrocarbon in gasoline. In particular, as the primary catalyst, N-hydroxyimide group-containing compounds each having an N-hydroxyimide group may be used. Among those compounds, N-hydroxyphthalimide (hereinafter referred to as “NHPI”) or a NHPI derivative has a significant function.

The auxiliary catalyst has a function to produce an alcohol by reduction of an alkyl hydroperoxide produced from an alkyl radical. In particular, as the auxiliary catalyst, transition metal compounds may be used. Among those compounds, a compound selected from the group consisting of a cobalt compound, a manganese compound, and a copper compound is preferably used. As the cobalt compound, for example, cobalt (II) acetate may be used; as the manganese compound, for example, manganese (II) acetate may be used; and as the copper compound, for example, copper (I) chloride may be used.

As a method for carrying the above primary catalyst and auxiliary catalyst onto the porous carriers, for example, a known impregnating method may be used. For example, after a slurry containing the primary catalyst and the auxiliary catalyst at a predetermined mixing ratio is prepared, small ball-shaped porous carriers are impregnated in the slurry thus prepared. Subsequently, after the porous carriers are recovered from the slurry, and an excessive slurry adhered to the surfaces of the porous carriers was removed, drying is performed under predetermined conditions. As a result, the reforming catalyst 152 in which the primary catalyst and the auxiliary catalyst are uniformly carried on the surfaces of the porous carriers is obtained.

Hereinafter, the reforming reaction performed in the reformer 15 will be described in detail.

First, as shown in the following reaction formula (1), the reforming reaction of this embodiment is started by a hydrogen abstraction reaction in which a hydrogen atom is abstracted from a hydrocarbon in gasoline to produce an alkyl radical. This hydrogen abstraction reaction is performed by functions of the primary catalyst, radicals, oxygen molecules, and the like.

RH→R. Chemical Formula (1)

[In the chemical formula (1), RH represents a hydrocarbon, and R. represents an alkyl radical.]

Next, as shown in the following chemical formula (2), the alkyl radical produced by the above hydrogen abstraction reaction is bonded to an oxygen molecule to produce an alkyl peroxy radical.

R.+O₂→ROO. Chemical Formula (2)

[In the chemical formula (2), O₂ represents an oxygen molecule, and ROO. represents an alkyl peroxy radical.]

Subsequently, as shown in the following reaction formula (3), the alkyl peroxy radical produced by the chemical formula (2) abstracts a hydrogen atom from a hydrocarbon contained in gasoline to produce an alkyl hydroperoxide.

ROO.+RH→ROOH+R. Chemical Formula (3)

[In the chemical formula (3), ROOH represents an alkyl hydroperoxide.]

Next, as shown in the following chemical formula (4), the alkyl hydroperoxide produced by the chemical formula (3) is reduced to an alcohol by the function of the auxiliary catalyst.

ROOH→ROH Chemical Formula (4)

[In the chemical formula (4), ROH represents an alcohol.]

In addition, as shown in the following chemical formula (5), the alkyl hydroperoxide produced by the chemical formula (3) is decomposed into an alkoxy radical and a hydroxy radical by the function of the auxiliary catalyst or heat.

ROOH→RO.+.OH Chemical Formula (5)

[In the chemical formula (5), RO. represents an alkoxy radical, and .OH represents a hydroxy radical.]

Subsequently, the alkoxy radical produced by the chemical formula (5) abstracts a hydrogen atom of a hydrocarbon contained in gasoline to produce an alcohol.

RO.+RH→ROH+R. Chemical Formula (6)

As described above, a hydrocarbon primarily contained in gasoline is reformed by oxidation into an alcohol. In more particular, since a hydrocarbon contained in gasoline includes hydrocarbons having 4 to 10 carbon atoms, the hydrocarbons are converted into alcohols having 4 to 10 carbon atoms. As described above, the fuel reforming system 1 according to this embodiment is configured to improve the octane number of gasoline.

Next, the condenser 16 is provided at a downstream side of the reformer 15 and at an upstream side of the filter mechanism 30 and separates a produced gas produced in the reformer 15 into a condensed phase primarily formed of a reformed fuel and a gas phase. The condenser 16 includes a heat exchanger not shown in the drawing, and the condensed phase primarily formed of a reformed fuel is separated from the gas phase by cooling the produced gas flowing out of an outlet of the reformer 15. In addition, as substances in the condensed phase, for example, by-products, such as benzoic acid and water, which will be described later, may be mentioned besides the alcohol as the main product, and as substances in the gas phase, for example, nitrogen, oxygen, and gas components of other by-products may be mentioned.

The reformed fuel tank 18 stores a reformed fuel in the condensed phase separated by the condenser 16. The reformed fuel tank 18 functions as a buffer tank temporarily stores an alcohol of the reformed fuel produced by reforming gasoline by the reformer 15.

In the fuel reforming system 1 according to this embodiment, in particular, the following reaction characteristically occurs.

That is, toluene, which is a representative aromatic component contained in gasoline, is oxidized by oxygen to produce benzoic acid through benzyl alcohol and benzaldehyde.

This reforming reaction is as shown by the following chemical formula (7).

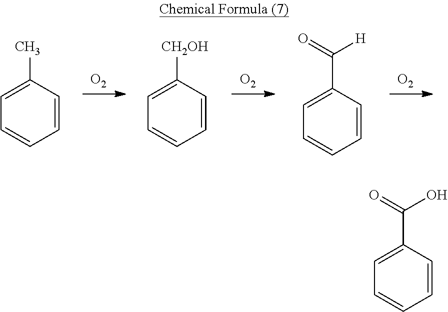

That is, in accordance with the reforming reaction by the reforming catalyst 152, an alcohol is produced as the main product, and as the by-product, benzoic acid is produced.

In this embodiment, in particular, the filter mechanism 30 including a filter formed of a sodium catalyst is inserted in a reformed fuel transport pipe 301 extending from the condenser 16 adjacent to the reformer 15 to the reformed fuel tank 18 so as to trap benzoic acid which is the by-product described above. In this case, since the condenser 16 is provided adjacent to the reformer 15, the amount of solid benzoic acid is increased. Hence, the trapping rate of benzoic acid by the filter is increased. This filter mechanism 30 will be further described in detail with reference to the drawing.

The reformed fuel supply portion 19 supplies a reformed fuel stored in the reformed fuel tank 18 into the cylinder of the engine by an injector. The reformed fuel supply portion 19 includes a reformed fuel pump 191, a reformed fuel supply pipe 192, and the injector not shown in the drawing. The reformed fuel supply portion 19 supplies the reformed fuel stored in the reformed fuel tank 18 into the cylinder of the engine not shown in the drawing through the reformed fuel supply pipe 192 and the injector by driving the reformed fuel pump 191. The supply amount of the alcohol is controlled by adjusting the amount injected from the injector using the ECU.

The gas phase supply portion 20 supplies a gas phase substance separated by the condenser 16 into a suction port of the engine. The gas phase supply portion 20 includes a gas phase supply pipe 201 connected to the suction port of the engine. The gas phase substance separated by the condenser 16 is supplied into the suction port of the engine through the gas phase supply pipe 201.

Hereinafter, the filter mechanism 30 inserted in the reformed fuel transport pipe 301 extending from the condenser 16 adjacent to the reformer 15 to the reformed fuel tank 18 will be described in detail.

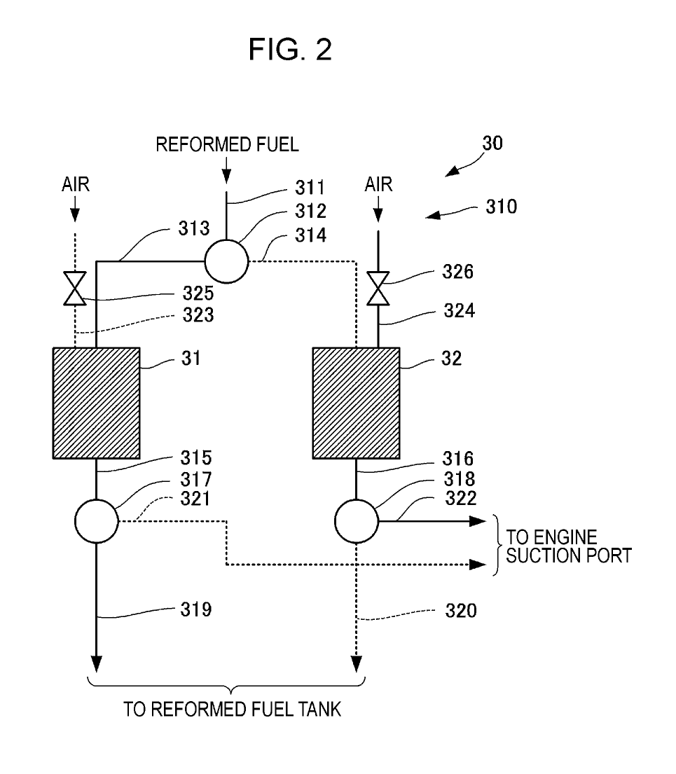

FIG. 2 is a schematic view showing one example of a filter mechanism applied to the fuel reforming system shown in FIG. 1.

The filter mechanism 30 shown in FIG. 2 includes a first filter 31, a second filter 32 having a similar structure to that thereof, and a flow path switching mechanism 310 switching between flow paths relating to the first filter 31 and the second filter 32.

The first filter 31 and the second filter 32 are each formed so that a sodium catalyst is carried on silica particles, and the silica particles carrying the sodium catalyst as described above are filled in a case. By the first filter 31 and the second filter 32 each having the structure as described above, a substance larger than a space formed between the closely packed silica particles can be trapped and removed.

The flow path switching mechanism 310 includes at an upstream side of the first filter 31 and the second filter 32, an inlet pipe line portion 311 which is a part of the reformed fuel transport pipe 301 and which is located immediately under the condenser 16, a first flow path switch 312 which is a three-way valve connected to the inlet pipe line portion 311 at a first port thereof, a first pipe line portion 313 connecting a second port of the first flow path switch 312 to the inlet of the first filter 31, and a second pipe line portion 314 connecting a third port of the first flow path switch 312 to the inlet of the second filter 32.

The flow path switching mechanism 310 further includes at a downstream side of the first filter 31, a third pipe line portion 315 directly connected to the outlet of the first filter 31, a second flow path switch 317 which is a three-way valve connected to the third pipe line portion 315 at a first port thereof, a fifth pipe line portion 319 connecting a second port of the second flow path switch 317 to the reformed fuel tank 18, and a seventh pipe line portion 321 connecting a third port of the second flow path switch 317 to the engine suction port (not shown). In addition, the flow path switching mechanism 310 also further includes at a downstream side of the second filter 32, a fourth pipe line portion 316 directly connected to the outlet of the second filter 32, a third flow path switch 318 which is a three-way valve connected to the fourth pipe line portion 316 at a first port thereof, a sixth pipe line portion 320 connecting a second port of the third flow path switch 318 to the reformed fuel tank 18, and an eighth pipe line portion 322 connecting a third port of the third flow path switch 318 to the engine suction port (not shown).

The seventh pipe line portion 321 and the eighth pipe line portion 322 located at a downstream side of the first filter 31 and the second filter 32, respectively, each form an introduction path introducing air containing phenol reformed by the sodium catalyst of the filter without modification into the corresponding suction port of the internal combustion engine.

In addition, the first filter 31 is provided at the inlet thereof with a first air introduction pipe 323 functioning to introduce air, and as is the case described above, the second filter 32 is provided at the inlet thereof with a second air introduction pipe 324 functioning to introduce air.

A first air valve 325 is inserted in the first air introduction pipe 323, and as is the case described above, a second air valve 326 is inserted in the second introduction pipe 324.

In FIG. 2, among the pipe lines in the flow path switching mechanism 310, a pipe line through which a fluid flows at a certain point of time is represented by a solid line, and a pipe line through which no fluid flows is represented by a dotted line.

The operation of the filter mechanism 30 described with reference to FIG. 2 will be described in detail using a flowchart after the overall operation of the fuel reforming system 1 is described in detail with reference to FIG. 1.

The fuel reforming system 1 according to this embodiment having the structure as described with reference to FIG. 1 is operated as follows by the control of the ECU.

First, in accordance with the operation condition of the engine, when gasoline is determined to be reformed, it is judged whether the temperature of the engine cooling water is a predetermined value or more or not. When the temperature of the engine cooling water is less than the predetermined value since the engine is immediately after driven, a reformed fuel stored in the reformed fuel tank 18 in previous reforming is supplied to the engine by the reformed fuel pump 191.

On the other hand, when the temperature of the engine cooling water is the predetermined value or more, the fuel valve 133 and the air valve 114 are opened. Subsequently, by the reforming pump 131, gasoline is pressure-transported into the mixer 14 from the fuel tank 12. At the same time, by the air pump 112, air passing through the air filter 111 is introduced into the mixer 14.

In the fuel reforming system 1 according to this embodiment, in the supply system 10, the air introduction portion 11 and the fuel introduction portion 13 are synchronously operated under the control of the ECU, and the air and the fuel to be supplied to the mixer 14 are controlled so that the rate of the fuel (gasoline) is 22 percent by mass or more.

In addition, under the control of the ECU, in order to obtain a desired and appropriate reforming reaction time, feedback control is performed on the degree of opening of each of the fuel valve 133 and the air valve 114 based on the gasoline flow rate monitored by the fuel flow meter 132 and the air flow rate monitored by the air flow meter 113.

Next, after the gasoline and the air introduced into the mixer 14 are uniformly mixed together to form a mixture while heated to a predetermined temperature, the mixture thus formed is supplied into the reformer 15. When the reactions represented by the above reaction formulas (1) to (6) proceed, a hydrocarbon which is a primary component of the gasoline in the mixture supplied into the reformer 15 is converted into an alcohol by the function of the reforming catalyst 152. In this step, based on the temperature monitored by the temperature sensor, the supply of the engine cooling water is controlled. As a result, the inside of the reformer 15 is maintained at a desired and appropriate temperature.

In addition, as the gasoline is progressively reformed by the function of the reforming catalyst 152, the reaction represented by the above reaction formula (7) also proceeds, so that an alcohol as a main product and benzoic acid as a by-product are produced.

Next, the gas produced by the reformer 15 is cooled by the heat exchanger in the condenser 16 and is separated into a condensed phase and a gas phase. In the separated condensed phase, besides the alcohol as the main product, the by-products, such as benzoic acid and water, are contained, and the reformed fuel thus separated is introduced through the reformed fuel transport pipe 301 into the reformed fuel tank 18 for storage.

In particular, in this embodiment, benzoic acid, which is the by-product, is trapped by the filter mechanism 30 inserted in the reformed fuel transport pipe 301, and the alcohol, which is the main product, passing through the filter mechanism 30 is stored in the reformed fuel tank 18.

The reformed fuel (alcohol) in the reformed fuel tank 18 is supplied to the injector by the reformed fuel pump 191 for combustion in the cylinder of the engine. On the other hand, the separated gas phase substance is supplied into the suction port of the engine.

In accordance with the operation condition of the engine, when the gasoline is determined not to be reformed, first, the air pump 112 is stopped, and the air valve 114 is closed, so that the supply of air into the mixer 14 is stopped. Next, after all air is discharged by the gasoline filled in the reformer 15, the reforming pump 131 is stopped, and the fuel valve 133 is closed, so that the supply of the gasoline into the mixer 14 is stopped. As a result, while the system is stopped, the case in which the reforming reaction proceeds by oxygen remaining in the reformer 15 can be avoided.

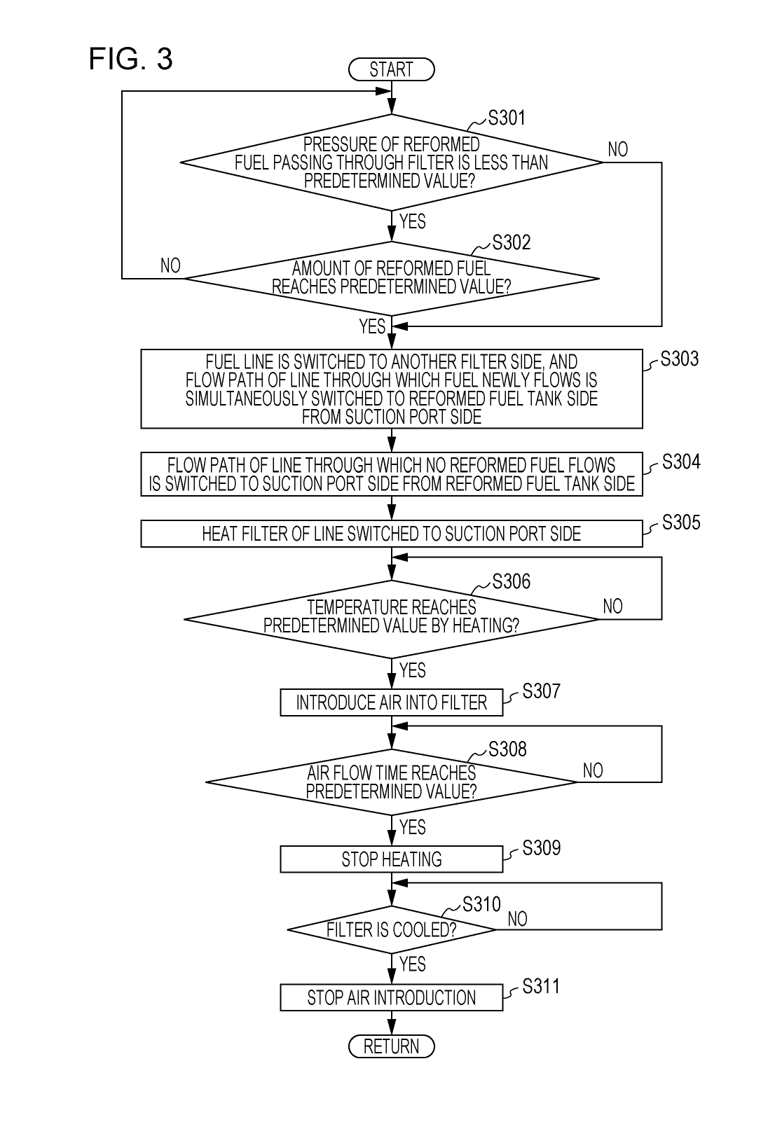

In this case, the filter mechanism 30 inserted in the reformed fuel transport pipe 301 has the structure shown in the above FIG. 2. The operation of the filter mechanism 30 as described above will be described with reference to FIG. 2 together with FIG. 3 which is a flowchart showing the operation of the filter mechanism 30.

In more detail, FIG. 3 is a flowchart sowing a filter regeneration operation performed when the filter is regenerated in the filter mechanism shown in FIG. 2.

That is, although the first filter 31 and the second filter 32 of the filter mechanism 30 each trap benzoic acid as described above, the filter is gradually clogged, and the resistance of the flow path is increased, so that the function of the filter is degraded. Hence, in this embodiment, a regeneration treatment as shown by the following reaction formula (8) is intermittently performed to recover the function of the filter.

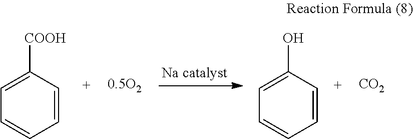

That is, the first filter 31 and the second filter 32, each of which is formed by filling silica particles carrying the sodium catalyst in the case as described above, are heated, and air is allowed to pass therethrough, so that phenol is produced.

A treatment procedure of the filter regeneration operation is as follows. The treatment by the following sequential steps is primarily performed, for example, by the ECU described above.

The pressure of the reformed fuel passing through the filter is always or periodically measured to monitor the condition of one of the first filter 31 and the second filter 32 in operation (Step S301). The detection point of this pressure is, for example, the inlet pipe line portion 311, and a pressure sensor (not shown) is provided at this point for detection.

When the pressure is less than a predetermined value (Step S301: YES), the filter is not clogged and normally functions, and hence the regeneration treatment is not required. Accordingly, judgment whether the amount of the reformed fuel reaches a predetermined value or not is then performed (Step S302).

In Step S302, in order to periodically perform the regeneration of the filter, the amount of the reformed fuel relating to the operation time of the filter is monitored. This monitoring is performed, for example, based on the detection value of a liquid level sensor (not shown) of the reformed fuel tank 18.

When the amount of the reformed fuel is judged to be less than the predetermined value in Step S302 (Step S302: NO), the operation time of the filter is not long enough to perform the regeneration treatment, and Step S301 is again performed.

When the pressure of the reformed fuel passing through the filter reaches the predetermined value in Step S301 (Step S301: NO), the filter is clogged, and in order to perform the regeneration treatment, Step S303 is then performed.

In addition, when the amount of the reformed fuel reaches the predetermined value in Step S302 (Step S302: YES), the operation time of the filter is long enough to perform the regeneration treatment, and in order to performed the regeneration treatment, Step S303 is then performed.

In Step S303, the supply line (pipe line) of the reformed fuel to one filter is switched to the supply line (pipe line) of the reformed fuel to the other filter. At the same time, a line (pipe line) at a downstream side of the filter through which the reformed fuel newly flows is switched from a suction port side to a reformed fuel tank side.

As for the filter mechanism 30 described with reference to FIG. 2, the above switching operation will be further described in detail.

Hereinafter, the state before the present state shown in FIG. 2 (the relationship between the solid line and the dotted line described above) will be described, that is, the state in which the first port of the first low switch 312 is communicated with the third port thereof, the first port of the second flow path switch 317 is communicated with the third port thereof, and the first port of the third flow path switch 318 is communicated with the second port thereof will be described.

In the state described above, the reformed fuel flows through a route starting from the inlet pipe line portion 311, the first flow path switch 312, the second pipe line portion 314, the second filter 32, the fourth pipe line portion 316, the third flow path switch 318, the sixth pipe line portion 320, to the reformed fuel tank 18 in this order.

On the other hand, in the state described above, no reformed fuel flows through a route starting from the first flow path switch 312, the first pipe line portion 313, the first filter 31, the third pipe line portion 315, the second flow path switch 317, the fifth pipe line portion 319, to the reformed fuel tank 18 in this order.

In the state described above, when it is detected that the pressure of the reformed fuel passing through the filter reaches the predetermined value in Step S301 (Step S301: NO), the probability of clogging of the second filter 32 is high. Hence, in Step S303, at the first flow path switch 312, the communication flow path is switched from the third port to the second port. As a result, the supply line of the reformed fuel to the filter is switched from the second pipe line portion 314 which is a supply line of the reformed fuel to the second filter 32 to the first pipe line portion 313 which is a supply line of the reformed fuel to the first filter 31.

At the same time, at the second flow path switch 317, the communication flow path is switched from the third port to the second port, and at the third flow path switch 318, the communication path is switched from the second port to the third port.

In Step S303, when the switching is performed as described above at each of the first flow path switch 312, the second flow path switch 317, and the third flow path switch 318, the flow path as shown by the solid line in FIG. 2 is formed.

That is, the reformed fuel flows through the route starting from the inlet pipe line portion 311, the first flow path switch 312, the first pipe line portion 313, the first filter 31, the third pipe line portion 315, the second flow path switch 317, the fifth pipe line portion 319, to the reformed fuel tank 18 in this order.

On the other hand, no reformed fuel flows through the route starting from the first flow path switch 312, the second pipe line portion 314, to the second filter 32 in this order.

Those described above are the operation of the filter mechanism 30 (flow path switching mechanism 310) of FIG. 2 in Step S303.

Next, the flow path of the line (pipe line) through which no reformed fuel flows by this switching is switched to the suction port side from the reformed fuel tank side (Step S304).

In the filter mechanism 30 shown in FIG. 2, in the route starting from the second pipe line portion 314, the second filter 32, the fourth pipe line portion 316, to the third flow path switch 318 through which no reformed fuel flows by switching the communication flow path at the first flow path switch 312, the communication flow path at the third flow path switch 318 is switched. By this switching, the communication flow path at the third flow path switch 318 is switched to the third port thereof, so that a flow path starting from the third flow path switch 318, the eighth pipe line portion 322, to the engine suction port (not shown) in this order is formed.

Next, the filter at a line (pipe line) side at which the third port of the third flow path switch 318 is communicated with the engine suction port side is heated (Step S305).

In the filter mechanism 30 shown in FIG. 2, the second filter 32 is heated by a heater (not shown).

As the result of the heating in Step S305, the temperature of the filter (the second filter 32 of the filter mechanism 30 in this case) is monitored by a temperature sensor (not shown) (Step S306) and is waited until reaching a predetermined value (Step S306: NO), and when the temperature reaches the predetermined value (Step S306: YES), air is introduced into the filter (Step S307).

In the filter mechanism 30 shown in FIG. 2, in Step S307, the second air valve 326 inserted into the second air introduction pipe 324 is opened, so that air is introduced.

Since the filter is heated, and air is allowed to flow therethrough, the reaction of the regeneration treatment represented by the reaction formula (8) proceeds.

After air is introduced into the filter (the second filter 32 of the filter mechanism 30 in this case) corresponding to that of Step S307, the flow time of the air is monitored (Step S308) and is waited until reaching a predetermined value (Step S308: NO). During this waiting time (Step S308: NO), the reaction of the regeneration treatment described above substantially continues.

While the regeneration treatment represented by the reaction formula (8) continues, phenol is produced from benzoic acid trapped by the filter.

In the filter mechanism 30 shown in FIG. 2, when phenol is produced from the filter as described above, in Step S304, the third port of the third flow path switch 318 already forms the flow path from the eighth pipe line portion 322 to the engine suction port (not shown).

As described above, the eighth pipe line portion 322 forms an introduction path introducing air containing phenol reformed by the sodium catalyst of the filter (second filter 32 in this case) without modification into the suction port of the internal combustion engine.

Accordingly, phenol having a high octane number is supplied to operate the engine, and the operation condition is effectively realized with a fuel having a high octane number.

when the flow time of air reaches the predetermined value (Step S308: YES), the heating is stopped (Step S309).

In the filter mechanism 30 shown in FIG. 2, power supply to the heater (not shown) of the second filter 32 is stopped.

After the heating in Step S309 is stopped, the temperature of the filter is monitored (Step S310) until the filter is cooled (Step S310: NO). When the filter is cooled (Step S310: YES), the introduction of air into the filter is stopped (Step S311).

In the filter mechanism 30 shown in FIG. 2, the temperature of the second filter 32 is monitored by a temperature sensor (not shown), and when the temperature is decreased to a predetermined value, the second air valve 326 inserted into the second air introduction pipe 324 is closed to stop the introduction of air.

At the stage described above, the regeneration treatment of the second filter 32 is completed, and the second filter 32 is ready to be changed with the first filter 31 in operation.

In the state described above, as described above in Step S301 and Step S302, when the pressure of the reformed fuel passing through the filter (the first filter 31 in this case) reaches the predetermined value, or the amount of the reformed fuel reaches the predetermined value, as described in Step S303 and the following Steps, the filter to be processed by the regeneration treatment is changed from the second filter 32 to the first filter 31.

From the first filter 31 described above which is changed from the second filter 32 and which is to be processed by the regeneration treatment, as described in Step S307 and Step S308, phenol is produced by the reaction of the reaction formula (8), and the phenol thus produced is supplied to the engine suction port (not shown) through the seventh pipe line portion 321 at this time.

As described above, the seventh pipe line portion 321 also forms an introduction path introducing air containing phenol reformed by the sodium catalyst of the filter without modification into the corresponding suction port of the internal combustion engine.

Hence, during the regeneration treatment performed on the first filter 31, phenol having a high octane number is also supplied to the operation of the engine, and the operation condition is effectively realized with a fuel having a high octane number.

As has thus been described with reference to FIG. 2, the flow path switching mechanism is a mechanism which switches the flow path structure such that when a plurality of filters are provided, and at least one filter thereof is placed in a first flow path structure which traps benzoic acid, at least one another filter of the plurality of filters is placed in a second flow path structure which reforms benzoic acid into phenol.

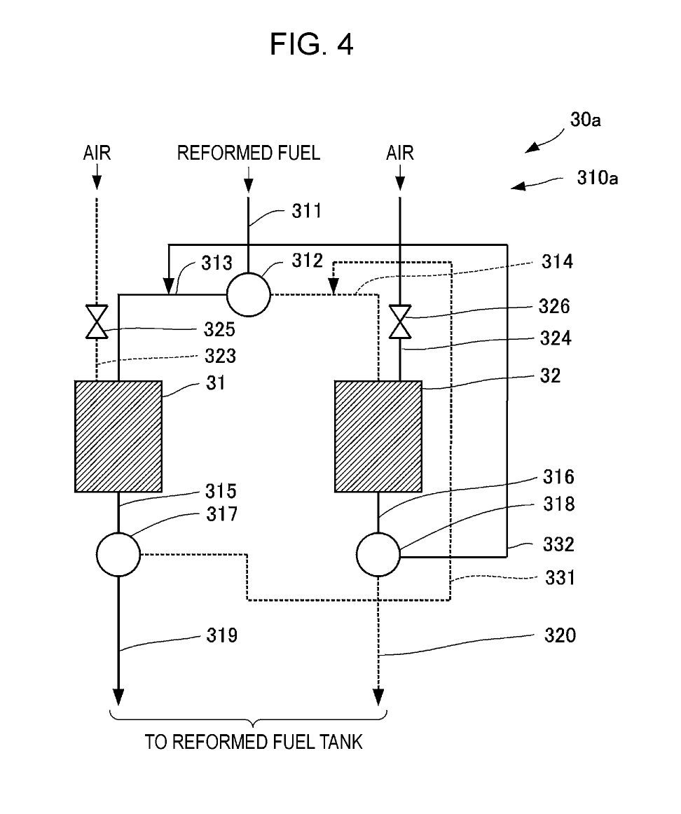

FIG. 4 is a schematic view showing a structural example of a filter mechanism according to another embodiment applied to the fuel reforming system shown in FIG. 1.

In FIG. 4, members corresponding to the members shown in the above FIG. 2 are designated by the same reference numerals, and detailed description thereof will be omitted.

The point different between a filter mechanism 30a shown in FIG. 4 and the filter mechanism 30 shown in FIG. 2 is that instead of the seventh pipe line portion 321 and the eighth pipe line portion 322 of the flow path switching mechanism 310 of the filter mechanism 30, a first reflux pipe 331 and a second reflux pipe 332 are provided to form a flow path switching mechanism 310a.

That is, in the filter mechanism 30, there are provided the seventh pipe line portion 321 and the eighth pipe line portion 322 each functioning as an introduction path introducing phenol itself into the corresponding suction port of the internal combustion engine, the phenol being produced by the reaction of the reaction formula (8) when the above regeneration treatment is performed on one of the first filter 31 and the second filter 32. On the other hand, in the filter mechanism 30a, there are provided the first reflux pipe 331 connecting the third port of the second flow path switch 317 located at a downstream side of the first filter 31 to a middle point of the second pipe line portion 314 located at an upstream side of the second filter 32 and the second reflux pipe 332 connecting the third port of the third flow path switch 318 located at a downstream side of the second filter 32 to a middle point of the first pipe line portion 313 located at an upstream side of the first filter 31.

In the filter mechanism 30a shown in FIG. 4, by the flow path switching mechanism 310a having the structure as described above, phenol which is produced by the reaction of the reaction formula (8) when the above regeneration treatment is performed on one of the first filter 31 and the second filter 32 is merged and mixed with the flow of the reformed fuel from the inlet pipe line portion 311. That is, phenol having a high octane number is mixed with the reformed fuel so as to further improve the octane number of the reformed fuel.

Although being approximately the same as that described with reference to the flowchart shown in FIG. 3, the filter regeneration operation performed during the filter regeneration in the filter mechanism 30a shown in FIG. 4 is slightly different therefrom.

That is, according to the filter regeneration operation in the filter mechanism 30a shown in FIG. 4, instead of performing Step S304 of the flowchart shown in FIG. 3, the flow path of the line through which no reformed fuel flows is switched to be merged with the flow of the reformed fuel from the inlet pipe line portion 311 by the first reflux pipe 331 or the second reflux pipe 332. The other points of the filter regeneration treatment are the same as those described with reference to the flowchart shown in FIG. 3.

In addition, the present disclosure is not limited to the embodiments described above and, for example, may be modified or improved within the scope of the present disclosure.

In the above embodiments, although gasoline is used as the fuel, the fuel is not limited thereto. For example, even in the case of using an alcohol-containing gasoline containing an alcohol such as ethanol, an effect similar to that described above can also be obtained.

In addition, as the flow path switching mechanism of the filter mechanism, the structure may also be formed in which a flow path directly supplying phenol produced by the filter regeneration treatment as a fuel into the corresponding cylinder of the internal combustion engine as described in the embodiment shown in FIG. 2 and a flow path merging the phenol thus produced with a main product by the reformer as described in the embodiment shown in FIG. 4 are both provided and are switchable to each other.

The present application was made in consideration of the situation described above and provides a fuel reforming system excellent in use efficiency of a raw fuel and in improvement in octane number.

In the present disclosure, the following technique is proposed.
(1) A fuel reforming system which reforms a fuel to be supplied into an internal combustion engine, includes:
a reformer (for example, a reformer 15 which will be described later) containing a reforming catalyst which reforms a fuel primarily formed of a hydrocarbon using air to produce an alcohol as a main product and benzoic acid as a by-product;
at least one filter (for example, a first filter 31 or a second filter 32 which will be described later) which includes a sodium catalyst, which is provided at a downstream side of the reformer, and which allows the alcohol as the main product produced by the reforming catalyst to pass therethrough and traps the benzoic acid precipitated as the by-product; and
at least one flow path (for example, a first reflux pipe 331 or a second reflux pipe 332 which will be described later) which merges phenol with the main product produced by the reformer or at least one flow path (for example, a seventh pipe line portion 321 or an eighth pipe line portion 322 which will be described later) which directly supplies phenol into the internal combustion engine as a fuel, the phenol being produced by reforming of the trapped benzoic acid with air which is allowed to pass through the filter.

According to the fuel reforming system of the above (1), since the raw fuel is not required to pass through the reformer a plurality of times, the use efficiency of the raw fuel is excellent, and in addition, since phenol having a significantly high octane number is obtained from the by-product produced by the reformer and is also used, an effective octane number can be further increased.

(2) The fuel reforming system of the above (1) may further include a condenser (for example, a condenser 16 which will be described later) which is provided at a downstream side of the reformer and at an upstream side of the filter and which forms a gas produced by the reformer into a condensed phase primarily formed of a reformed fuel and also precipitates the benzoic acid.

According to the fuel reforming system of the above (2), in particular in the fuel reforming system of the above (1), since solid benzoic acid is increased, the trapping rate by the filter is increased. As a result, the octane number can be further increased.

(3) The fuel reforming system of the above (1) or (2) may further include a flow path switching mechanism (for example, a flow path switching mechanism 310 which will be described later) which switches a flow path structure such that when the filters are provided, and at least one filter (for example, the first filter 31 which will be described later) thereof is disposed in a first flow path structure (for example, a route starting from an inlet pipe line portion 311, a first flow path switch 312, a first pipe line portion 313, the first filter 31, a third pipe line portion 315, a second flow path switch 317, a fifth pipe line portion 319, to a reformed fuel tank 18, each of which will be described later) which traps the benzoic acid, at least one another filter (for example, the second filter 32 which will be described later) of the filters is disposed in a second flow path structure (for example, a route starting from a second air introduction pipe 324 (second air valve 326), the second filter 32, a fourth pipe line portion 316, a third flow path switch 318, an eighth pipe line portion 322, to an engine suction port, each of which will be described later) which reforms the benzoic acid into the phenol.

According to the fuel reforming system of the above (3), in particular in the fuel reforming system of the above (1) or (2), the benzoic acid generated in a reformed fuel from the reformer can always be trapped. As a result, the octane number can be further increased.

(4) The fuel reforming system of one of the above (1) to (3) may further include: at least one introduction path (for example, the seventh pipe line portion 321 or the eighth pipe line portion 322, which will be described later) directly introducing air containing the phenol reformed by the sodium catalyst of the filter without modification to the suction port of the internal combustion engine.

According to the fuel reforming system of the above (4), in particular in the fuel reforming system of one of the above (1) to (3), since the air containing the phenol reformed by the sodium catalyst is used without modification as a fuel for the internal combustion engine, a knocking reduction effect can be increased without providing any special mechanisms.

According to the present disclosure, a fuel reforming system which is excellent in use efficiency of a raw fuel and which obtains a fuel having a significantly high octane number can be realized.

Obviously, numerous modifications and variations of the present invention are possible in light of the above teachings. It is therefore to be understood that within the scope of the appended claims, the invention may be practiced otherwise than as specifically described herein.

What is claimed is:

1. A fuel reforming system which reforms a fuel to be supplied into an internal combustion engine, the system comprising:
a reformer containing a reforming catalyst which reforms a fuel primarily formed of a hydrocarbon using air to produce an alcohol as a main product and benzoic acid as a by-product;
at least one filter which includes a sodium catalyst, which is provided at a downstream side of the reformer, and which allows the alcohol as the main product produced by the reforming catalyst to pass therethrough and traps the benzoic acid precipitated as the by-product; and
at least one flow path which merges phenol with the main product produced by the reformer or at least one flow path which directly supplies phenol into the internal combustion engine as a fuel, the phenol being produced by reforming of the trapped benzoic acid with air which is allowed to pass through the filter.

2. The fuel reforming system according to claim 1, further comprising:
a condenser which is provided at a downstream side of the reformer and at an upstream side of the filter and which forms a gas produced by the reformer into a condensed phase primarily formed of a reformed fuel and also precipitates the benzoic acid.

3. The fuel reforming system according to claim 1, further comprising:
a flow path switching mechanism which switches a flow path structure such that when the filters are provided, and at least one filter thereof is disposed in a first flow path structure which traps the benzoic acid, at least one another filter is disposed in a second flow path structure which reforms the benzoic acid into the phenol.

4. The fuel reforming system according to claim 1, further comprising:
at least one introduction path introducing air containing the phenol reformed by the sodium catalyst of the filter without modification to a suction port of the internal combustion engine.

5. A fuel reforming system to reform a fuel to be supplied to an internal combustion engine, the system comprising:
a reformer including a reforming catalyst to reform a fuel comprising a hydrocarbon to produce an alcohol and benzoic acid as a by-product using air;
a first filter including a sodium catalyst to trap the benzoic acid produced by the reforming catalyst, the alcohol produced by the reforming catalyst passing through the first filter, air passing through the first filter to reform the benzoic acid to phenol; and
a first flow path via which the phenol is mixed with the alcohol produced by the reforming catalyst or via which the phenol is directly supplied to the internal combustion engine as a fuel.

6. The fuel reforming system according to claim 5, further comprising:
a condenser which is provided between the reformer and the first filter and which forms a gas produced by the reformer into a condensed phase comprising a reformed fuel and also precipitates the benzoic acid.

7. The fuel reforming system according to claim 5, further comprising:
a second filter including a sodium catalyst to trap the benzoic acid produced by the reforming catalyst, the alcohol produced by the reforming catalyst passing through the second filter, the air passing through the second filter to reform the benzoic acid to the phenol;
a second flow path via which the phenol is mixed with the alcohol produced by the reforming catalyst or via which the phenol is directly supplied to the internal combustion engine; and
a flow path switching mechanism which switches a flow path structure such that when the first filter is disposed in a first flow path structure in which the first filter traps the benzoic acid, the second filter is disposed in a second flow path structure in which the second filter reforms the benzoic acid into the phenol.

8. The fuel reforming system according to claim 5, further comprising:
at least one introduction path introducing air containing the phenol reformed by the sodium catalyst of the first filter to a suction port of the internal combustion engine.
